(19) United States
(12) Patent Application Publication
(10) Pub. No.: US 2012/0052460 A1
(43) Pub. Date: Mar. 1, 2012

(54) DENTAL HANDPIECE SWIVEL COUPLING WITH AN AUTOCLAVABLE ILLUMINATOR ASSEMBLY

(75) Inventor: Luther GATES, Honey Brook, PA (US)

(73) Assignee: DENTALEZ, INC., Malvern, PA (US)

(21) Appl. No.: 12/871,325

(22) Filed: Aug. 30, 2010

(51) Int. Cl. A61B 1/24

(52) U.S. Cl. 433/29

(57) ABSTRACT

A swivel coupling for coupling a dental handpiece to a dental-handpiece hose assembly. An optical transmission element extends from the chamber. An illuminator assembly is removably retained in the chamber. The illuminator assembly has an emitter in register with the optical transmission element. The emitter is mounted on a substrate having an anode pad and a cathode pad electrically connected to the emitter. A first differential-thermal-expansion compliant conductor is in continuous electrical contact with the anode pad and an anode pin. A second differential-thermal-expansion compliant conductor is in continuous electrical contact with the cathode pad and a cathode pin. A heat sink is thermally coupled to the substrate. A differential-thermal-expansion load applicator is operatively coupled to the substrate and to a surface of the chamber. The differential-thermal-expansion load applicator is configured to maintain a thermal interface between the substrate and the heat sink in continuous compression.

### BACKGROUND OF THE INVENTION

The present invention relates to a dental handpiece swivel coupling having an autoclavable illuminator assembly. More particularly, the present invention relates to an autoclavable illuminator assembly removably retainable in a chamber of a dental handpiece swivel coupling having an optical transmission element terminating in the chamber and an exhaust tube configured to discharge an exhaust fluid into the chamber.

The conventional dental drill assembly 10 comprises a dental handpiece 12, a swivel coupling 14 and a hose assembly 16 having a tubing bundle 18 as shown in FIG. 1. The typical dental handpiece 12 has a head 20 supporting a high-speed air-driven turbine (not shown) to which a dental burr or drill bit 22 is attachable. The head 20 has multiple ports 24 proximal to the drill bit 22 to allow illumination and irrigation of a drill site. The body 26 of the handpiece 12 houses tubes (not shown) providing air to drive the turbine and water to irrigate the drill site. An exhaust tube (not shown) for removing the exhaust air from the turbine and water vapor entrained therein and an optical transmission element (not shown), typically a glass rod or an optical fiber bundle, are also housed in the handpiece body.

The swivel coupling 14 is provided to connect the tubing in the handpiece 12 to corresponding tubing in the tubing bundle 18 of the hose assembly 16 in a manner that allows the dental handpiece 12 to be essentially torque free as a dentist twists, rotates or otherwise manipulates the handpiece to orient the head of the handpiece as required to accomplish the drilling objective.

The typical swivel coupling 14, shown in an exploded perspective view in FIG. 2, has a nose 28 with O-ring seals 30 configured for slip fit insertion in a corresponding receiver 26a in the body 26 of the dental handpiece 12 in a manner that provides for alignment and mating of the tubes and an optical transmission element in the hand piece 12 with one end of the corresponding fluid and optical passageways in the swivel coupling 14. The other end of the fluid passageways mate with corresponding tubing in the hose assembly 16. The other end of the optical pathway terminates in register with an emitter 32, such as the xenon-halogen emitter of an illuminator assembly 34 removably retained in a first chamber 36 in a manifold (not shown) in the swivel coupling 14. Power is provided to the emitter 32 through a pair of conductor pins 38 that mate with electrical conductors in the hose assembly 16.

A second chamber (not shown) in the manifold removably retains a water valve assembly 40. The illuminator assembly 34 and the water valve assembly 40 are accessible when a threaded coupler 42 and rear seal 44 are removed. In addition to providing access to the first and second chambers, the threaded coupler 42 connects the swivel coupler 14 with the hose assembly 16.

Known limitations of conventional swivel couplings include the use of xenon-halogen lamps in the illuminator assembly. The lamps should be removed before the assembly is autoclaved. Further, the lamps are optically inferior emitters when compared to light emitting diodes (LEDs).

The substitution of an optical semiconductor device, such as an LED assembly for a halogen lamp as disclosed in U.S. Pat. No. 5,908,295 (Kawata), incorporated in its entirety herein by reference, improves the optical performance of the illuminator assembly but does not resolve the autoclave issues as the typical materials from which optical semiconductor devices and the components of a conventional swivel coupling are fabricated have disparate coefficients of thermal expansion. During repeated exposure to autoclave thermal cycles, differential thermal expansion degrades the integrity and continuity of the thermal and electrical interfaces between components rendering the swivel coupling unreliable and inoperative.

Accordingly, there is a compelling need for an illuminator assembly for a dental handpiece swivel coupling that is able to withstand repeated autoclave thermal cycles without the loss of electrical and thermal conductivity between component parts.

### BRIEF SUMMARY OF THE INVENTION

Briefly stated, one embodiment of the present invention is directed to a swivel coupling for coupling a dental handpiece to a dental-handpiece hose assembly comprising a housing having a first end configured to removably and swivelably couple to the dental handpiece and a second end configured to removably couple to the dental-handpiece hose assembly. A chamber is in the housing. An optical transmission element extends from the chamber. An illuminator assembly is removably retained in the chamber. The illuminator assembly comprises an emitter in register with the optical transmission element, the emitter mounted on a substrate having an anode pad and a cathode pad electrically connected to the emitter, an anode pin, a first differential-thermal-expansion compliant conductor in continuous electrical contact with the anode pad and the anode pin, a cathode pin, a second differential-thermal-expansion compliant conductor in continuous electrical contact with the cathode pad and the cathode pin, a heat sink thermally coupled to the substrate and a differential-thermal-expansion load applicator operatively coupled to the substrate and to a surface of the chamber, the differential-thermal-expansion load applicator configured to maintain a thermal interface between the substrate and the heat sink in continuous compression.

Another embodiment of the present invention is directed to an autoclavable illuminator assembly removably retainable in a chamber of a dental handpiece swivel coupling having an optical transmission element terminating in the chamber and an exhaust tube configured to discharge an exhaust fluid into the chamber. The illuminator assembly comprises an emitter mounted on a substrate having an anode pad and a cathode pad electrically connected to the emitter, an anode pin, a first differential-thermal-expansion compliant conductor in continuous electrical contact with the anode pad and the anode pin, a cathode pin, a second differential-thermal-expansion compliant conductor in continuous electrical contact with the cathode pad and the cathode pin, a heat sink thermally coupled to the substrate, and a differential-thermal-expansion load applicator operatively coupled to the substrate and configured to maintain a thermal interface between the substrate and the heat sink in continuous compression when the illuminator assembly is removably retained in the chamber and the emitter is in register with the optical transmission element.

Still another embodiment of the present invention is a swivel coupling for coupling a dental handpiece to a dental-handpiece hose assembly comprising a housing having a first end configured to removably and swivelably couple to the dental handpiece and a second end configured to removably couple to the dental-handpiece hose assembly. A chamber is in the housing. An illuminator assembly is removably retained in the chamber. The illuminator assembly comprises an emitter mounted on a substrate having an anode pad and a cathode pad electrically connected to the emitter. A first differential-thermal-expansion compliant conductor is in continuous electrical contact with the anode pad. A second differential-thermal-expansion compliant conductor is in continuous electrical contact with the cathode pad. A heat sink is thermally coupled to the substrate. A differential-thermal-expansion load applicator is operatively coupled to the substrate and to a surface of the chamber. The differential-thermal-expansion load applicator is configured to maintain a thermal interface between the substrate and the heat sink in continuous compression.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is an exploded perspective view of a prior art dental drill assembly;

FIG. 2 is an exploded perspective view of the prior art swivel coupling of the dental drill assembly of FIG. 1;

FIG. 3 is a side view of a dental handpiece swivel coupling in accordance with a preferred embodiment of the present invention;

FIG. 4 is a side cross-sectional view of the swivel coupling of FIG. 3;

FIG. 5 is a side perspective view of a portion of the swivel coupling of FIG. 3 showing in an exploded side perspective view an illuminator assembly in accordance with a preferred embodiment of the present invention;

FIG. 6 is a perspective view of the illuminator assembly of FIG. 4 showing the first and second differential-thermal-expansion compliant conductors in phantom;

FIG. 7 is a top perspective view of the emitter of the illuminator assembly of FIG. 6;

FIG. 8 is a bottom plan view of the emitter of FIG. 7;

FIG. 9 is a side elevation view of the emitter of FIG. 7;

FIG. 10 is an electrical schematic for the emitter of FIG. 7.

FIG. 11 is a top plan view of the heat sink of the illuminator assembly of FIG. 6;

FIG. 12 is a side perspective view of an anode pin of the illuminator assembly of FIG. 6; and

FIG. 13 is a perspective view of another preferred embodiment of an illuminator assembly in accordance with the present invention, showing another preferred embodiment of the first differential-thermal-expansion compliant conductor and another preferred embodiment second differential-thermal-expansion compliant conductor.

### DETAILED DESCRIPTION OF THE INVENTION

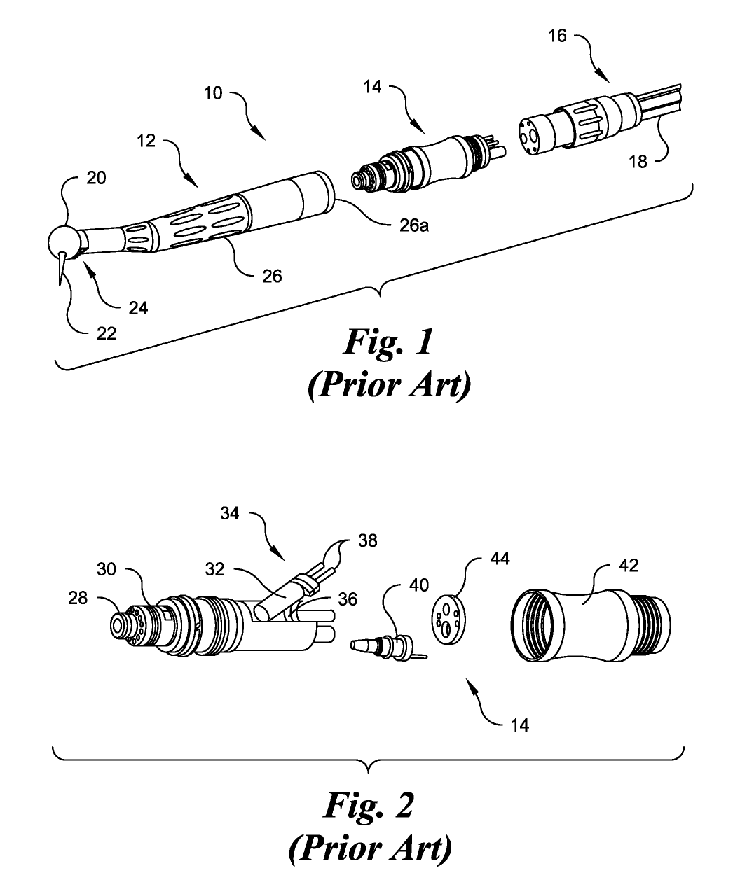

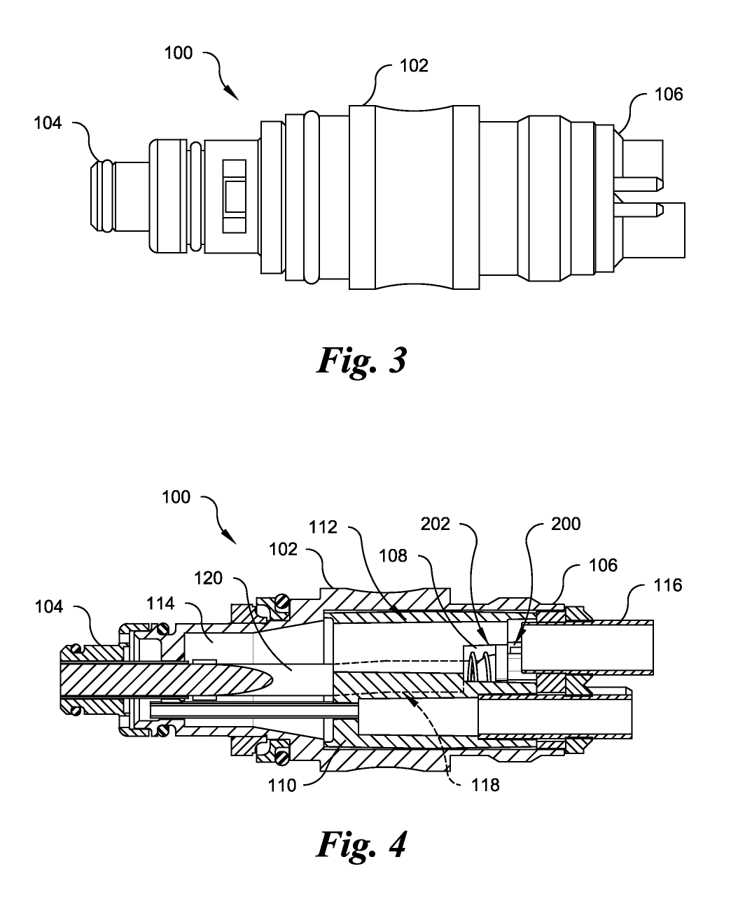

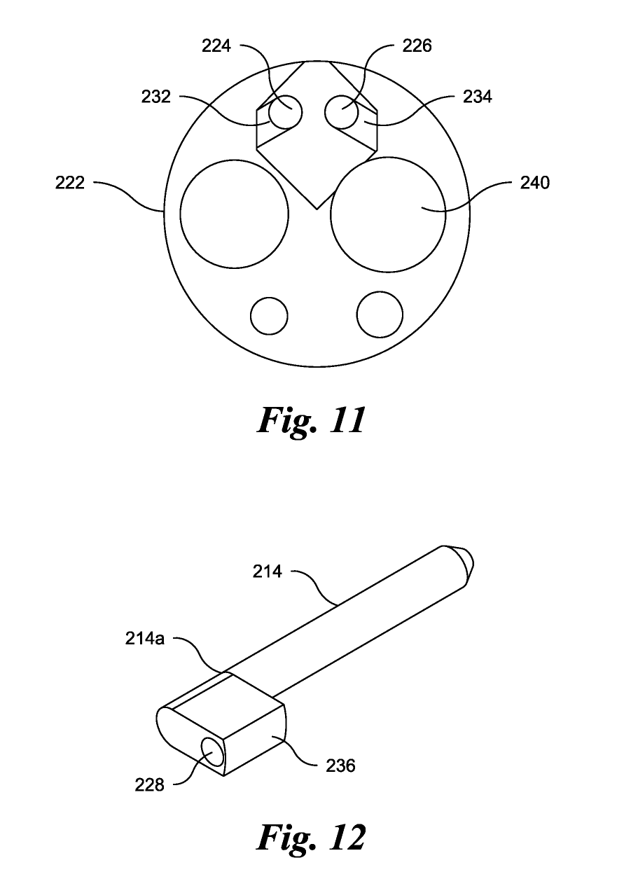

Referring to the drawings in detail, where like numerals indicate like elements throughout, there is shown in FIGS. 3-12 a first preferred embodiment of a dental handpiece swivel coupling, generally designated 100, and hereinafter referred to as the “swivel coupling” 100 in accordance with the present invention. The swivel coupling 100 is for coupling a dental handpiece to a dental-handpiece hose assembly, such as the dental handpiece 12 and dental-handpiece hose assembly 16 shown in FIG. 1.

The swivel coupling 100 comprises a housing 102 having a first end 104 configured to removably and swivelably couple to a dental handpiece and a second end 106 configured to removably couple to a dental-handpiece hose assembly. A chamber 108 is in the housing 102. In some embodiments, the chamber 108 may be proximal to the second end 106 of the housing 102. In other embodiments, the chamber 108 may be proximal to the first end 104 of the housing 102. In some embodiments, the chamber 108 may be in a manifold 110 through which a plurality of tubes (or channels) pass connecting supply and return tubes in a dental hose assembly with corresponding tubes in a dental handpiece. Typically, the tubes supply air and water to the handpiece to drive a turbine and irrigate the drill site.

The swivel coupling 100 also has an exhaust tube 114 configured to discharge an exhaust fluid into the chamber 108 and an exhaust discharge tube 116 that extends from the chamber 108. The exhaust tube 114, the chamber 108 and the exhaust discharge tube 116 are in fluid communication and form a fluid passageway 112. Exhaust fluid from the dental handpiece passes through the swivel coupling 100 in a fluid passageway 112.

An optical path 118 terminating in the chamber 108 extends from the chamber 108 to the first end 104 of the housing 102. The optical transmission path 118 may comprise any light propagating medium. In one embodiment, the path 118 is an optical transmission element 120 that is typically a fiber optic bundle or glass rod.

Referring to FIGS. 4-6, an autoclavable illuminator assembly 200 is removably retainable in the chamber 108. The illuminator assembly 200 comprises an emitter 202 in register with the optical path 118 when the illuminator assembly 200 is removably retained in the chamber 108. In some embodiments, the emitter 202 in register with the optical transmission element 120 when the illuminator assembly 200 is removably retained in the chamber 108. The emitter 202 desirably is an optical semiconductor device and preferably is a light emitting diode. In some embodiments, the emitter may include more than one light source.

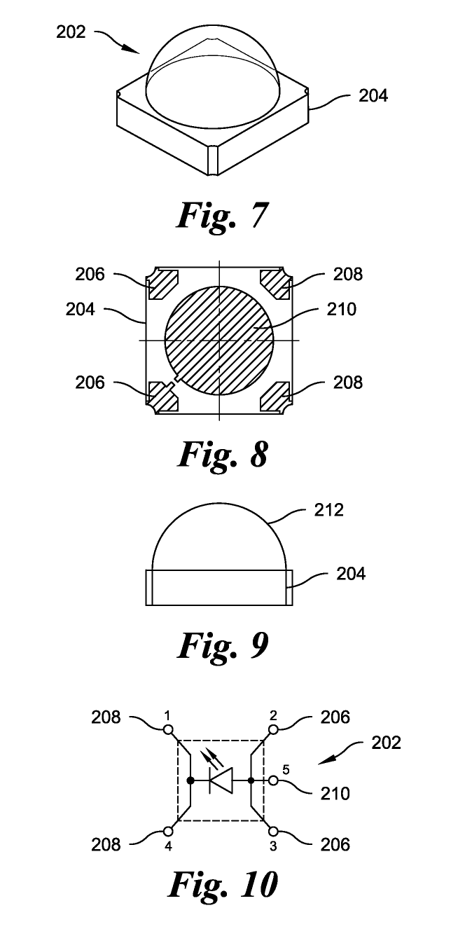

Referring to FIGS. 7-10, the emitter 202 is mounted on a substrate 204 having anode pads 206 and cathode pads 208 electrically connected to the emitter 202. A thermal contract pad 210 provided on the bottom of the substrate 204 is electrically connected to the anode pads 206. A protective lens 212 covers the emitter 202 and is mounted directly to the substrate 204.

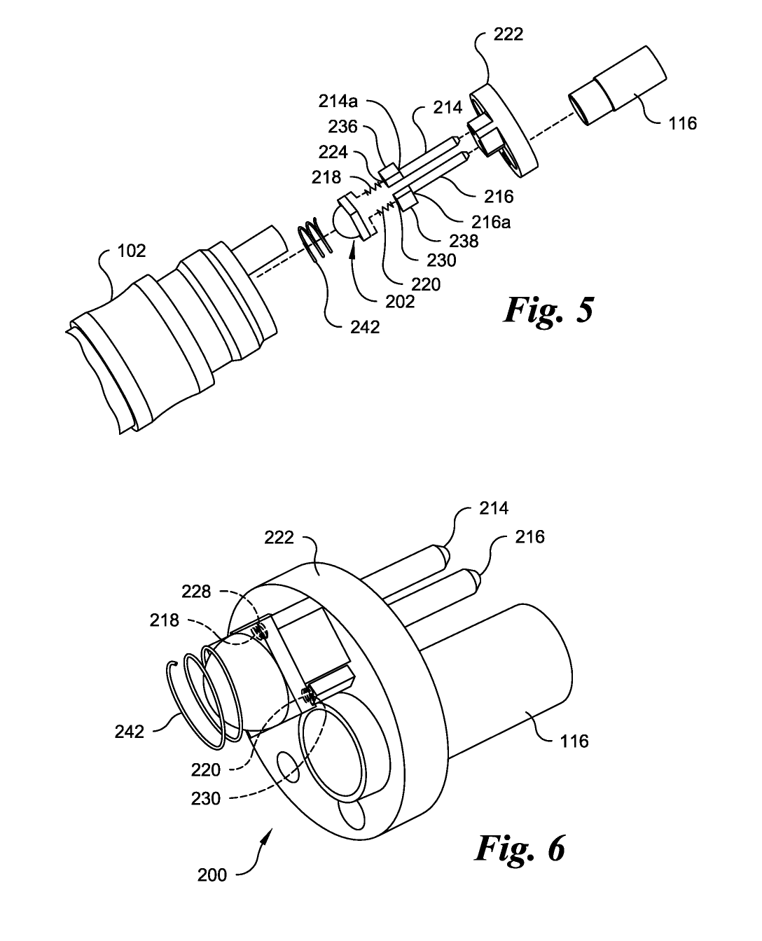

Referring to FIGS. 5, 6 and 12, the illuminator assembly 200 further comprises a first differential-thermal-expansion (DTE) compliant conductor 218 is in continuous electrical contact with the anode pad 206 and a second DTE compliant conductor 220 in continuous electrical contact with the cathode pad 208

In one embodiment, the first DTE compliant conductor 218 is a first compression spring under continuous compression applying a first spring force to one of the anode pads 206 and an anode pin 214 and the second DTE compliant conductor 220 is a second compression spring under continuous compression applying a second spring force to one of the cathode pads 208 and cathode pin 216. Preferably, the first and second compression springs are coil springs fabricated from a material such a cobalt chromium-nickel alloy commonly known as Elgiloy®. Alternative materials that are electrically conductive such as stainless steel may also be used. The DTE compliant conductors 218, 220 are not limited to coil springs. Other types of springs such as disc springs, conical compression washer and the like also may be used.

A heat sink 222 is thermally coupled to the substrate 204, desirably by an adhesive and preferably by a thermally conductive epoxy. In one embodiment, the adhesive is in continuous compression.

Referring to FIGS. 5, 6 and 11, in one embodiment, the heat sink 222 has an anode-pin heat-sink bore 224 through which the anode pin 214 extends and a cathode-pin heat-sink bore 226 through which the cathode pin extends. A first end 214a of the anode pin 214 is proximal to and spaced from the anode pad 206. A first anode-pin bore 228 in the first end 214a of the anode pin 214 supports the first DTE compliant conductor 218. A first end 216a of the cathode pin 216 is proximal to and spaced from the cathode pad 208. A first cathode-pin bore 230 in the first end 216a of the cathode pin 216 supports the second DTE compliant conductor 220.

In some embodiments, the anode-pin heat-sink bore 224 has a radially outwardly extending anode-pin heat-sink bore slot 232 and the cathode-pin heat-sink bore 226 has a radially outwardly extending cathode-pin heat-sink bore slot 234. The first end 214a of the anode pin 214 has a radially outwardly extending anode-pin tab 236 in the anode-pin heat-sink bore slot 232 and the first end 216a of the cathode pin 216 has a radially outwardly extending cathode-pin tab 238 in the cathode-pin heat-sink bore slot 236.

In addition to the anode-pin heat-sink bore 224 and the cathode-pin heat-sink bore 226, the heat sink 222 may have an exhaust discharge-tube heat-sink bore 240 through which the exhaust discharge tube 116 extends. In such embodiments, the emitter 202 is cooled by an exhaust fluid when the illuminator assembly 200 is retained in the chamber 108 and the exhaust fluid flows from the dental handpiece flows through the fluid passageway 112.

The illuminator assembly 200 further comprises a DTE load applicator 242 operatively coupled to the substrate 204 and to a surface 108a of the chamber 108. The DTE load applicator 242 is configured to maintain a thermal interface between the substrate 204 and the heat sink 222 in continuous compression when the illuminator assembly 200 is removably retained in the chamber 108 of the housing 102 of the swivel coupling 100 and the emitter 202 is in register with the optical transmission element 118. In one embodiment, the DTE load applicator 242 is a third compression spring under continuous compression applying a third spring force to the substrate 204 when the illuminator assembly 200 is removably retained in the chamber 108. As stated above, the third compression spring is not limited to a coil spring and may be any other type of spring such as a disc spring, a conical compression washer and the like. The third spring force applied by the DTE load applicator 242 to the substrate 204 is greater than the combination of the first spring force and the second spring force. The DTE load applicator 242 does not function as an electrical conductor. Accordingly, the materials from which the DTE load applicator 242 may be fabricated are not limited to electrically conductive materials.

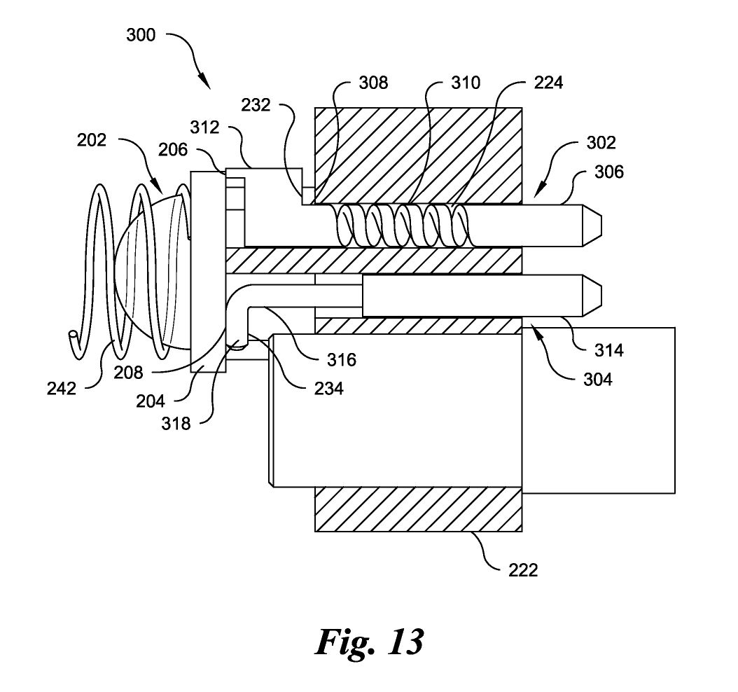

Referring to FIG. 13, another embodiment of an illuminator assembly generally designated 300, and hereinafter referred to as the “illuminator assembly” 300 in accordance with the present invention is shown in FIG. 13. With the exception of the DTE compliant conductors, the illuminator assembly 300 has substantially the same features as the illuminator assembly 200 discussed above. Although the common features are not discussed below for brevity, in FIG. 13 the features common to the illuminator assembly 200 and the illuminator assembly 300 are indicated by the same reference numbers.

The illuminator assembly 300 has a first DTE compliant conductor 302 and a second DTE compliant conductor 304. The first and second DTE compliant conductors 302, 304 are fabricated as single, continuous piece-parts formed from an electrically conducting material able to apply a spring force to the anode and cathode pads 206, 208 of the emitter 202 sufficient to maintain continuous electrical contact with the anode and cathode pads 206, 208 after repeated exposure to sterilizing autoclave thermal cycles.

The first DTE compliant conductor 302 has a first anode conductor end portion 306 distal to the anode pad 206, a second anode conductor end portion 308 in continuous contact with the anode pad 206 and an anode conductor mid-portion 310 between the first and second anode conductor end portions 306, 308. The first anode conductor end portion 306 has a generally pin-like shape configured to mate with a corresponding bore (not shown) in a dental hose assembly, such as the dental hose assembly 16 (see FIG. 1). The second anode conductor end portion 308 has a generally circular cross-section and a radially outwardly extending anode conductor tab 312 configured for insertion in the radially outwardly extending anode-pin heat-sink bore slot 232. The anode conductor mid-portion 310 is compressible relative to the first and second anode conductor end portions 306, 308 and applies a first anode conductor spring force to the first anode conductor end portion 306 and to the second anode conductor end portion 308, which, in turn, applies the first anode conductor spring force to the anode pad 206. In one embodiment, the anode conductor mid-portion 310 may be configured as a coil spring. In another embodiment, the anode conductor mid-portion may be configured as a rod having a smaller diameter than the diameter of the first anode conductor end portion 306. The diameter of the first anode conductor end portion 306 adjacent the mid-portion 310 is sized for an interference (or compression) fit in the anode-pin heat-sink bore 224. The diameter of the second anode conductor end portion 308 is sized for a sliding fit within the anode-pin heat-sink bore 224.

The second DTE compliant conductor 304 has a first cathode conductor end portion 314 distal to the cathode pad 208 and a second cathode conductor end portion 316 in continuous contact with the cathode pad 208. The first cathode conductor end portion 314 has generally pin-like shape configured to mate with a corresponding bore (not shown) in a dental hose assembly. The second cathode conductor end portion 316 has a circular cross-section and a radially outwardly extending cathode conductor tab 318 configured for insertion in the radially outwardly extending cathode-pin heat-sink bore slot 234. The diameter of the first cathode conductor end portion 314 is sized for a press (or compression) fit in the cathode-pin heat-sink bore 226. The diameter of the second cathode conductor end portion 316 is sized for a sliding fit within the cathode-pin heat-sink bore 234. The second cathode conductor end portion 316 is compressible relative to the first cathode conductor end portion 314 and applies a first cathode conductor spring force to the cathode pad 208 and the first cathode conductor end portion 314. In one embodiment, the second cathode conductor end portion 316 may be configured as a flexible rod. Alternatively, the second cathode conductor end portion 316 may be configured as a leaf spring.

The third spring force applied by the DTE load applicator 242 to the substrate 204 is greater than the combination of the first anode conductor spring force and the first cathode conductor spring force.

In one embodiment of the illuminator assembly 300, the second DTE compliant conductor 304 may be replaced with another first DTE compliant conductor 302. In another embodiment of the illuminator assembly 300, the first DTE compliant conductor 302 may be replaced with another second DTE compliant conductor 304.

Those skilled in the art will appreciate that changes could be made to the embodiments described above without departing from the broad inventive concept thereof. Therefore, the electronic learning device with a graphic user interface for interactive writing is not limited to the particular embodiments disclosed, but it is intended to cover modifications within the spirit and scope of the present invention as defined by the appended claims.

We claim:

1. A swivel coupling for coupling a dental handpiece to a dental-handpiece hose assembly comprising:
a housing having a first end configured to removably and swivelably couple to the dental handpiece and a second end configured to removably couple to the dental-handpiece hose assembly;
a chamber in the housing;
an optical transmission element extending from the chamber;
an illuminator assembly removably retained in the chamber, the illuminator assembly comprising:
an emitter in register with the optical transmission element, the emitter mounted on a substrate having an anode pad and a cathode pad electrically connected to the emitter;
an anode pin;
a first differential-thermal-expansion compliant conductor in continuous electrical contact with the anode pad and the anode pin;
a cathode pin;
a second differential-thermal-expansion compliant conductor in continuous electrical contact with the cathode pad and the cathode pin;
a heat sink thermally coupled to the substrate; and
a differential-thermal-expansion load applicator operatively coupled to the substrate and to a surface of the chamber, the differential-thermal-expansion load applicator configured to maintain a thermal interface between the substrate and the heat sink in continuous compression.

2. A swivel coupling of claim 1, further comprising a fluid passageway formed by an exhaust tube and an exhaust discharge tube in fluid communication with the chamber and the emitter is cooled by a fluid when the fluid flows through the fluid passageway.

3. An autoclavable illuminator assembly removably retainable in a chamber of a dental handpiece swivel coupling having an optical transmission element terminating in the chamber, the illuminator assembly comprising:
an emitter mounted on a substrate having an anode pad and a cathode pad electrically connected to the emitter;
an anode pin;
a first differential-thermal-expansion compliant conductor in continuous electrical contact with the anode pad and the anode pin;
a cathode pin;
a second differential-thermal-expansion compliant conductor in continuous electrical contact with the cathode pad and the cathode pin;
a heat sink thermally coupled to the substrate; and
a differential-thermal-expansion load applicator operatively coupled to the substrate and configured to maintain a thermal interface between the substrate and the heat sink in continuous compression when the illuminator assembly is removably retained in the chamber and the emitter is in register with the optical transmission element.

4. The illuminator assembly according to claim 3, wherein the first differential-thermal-expansion compliant conductor is a first compression spring under continuous compression and the second differential-thermal-expansion compliant conductor is a second compression spring under continuous compression.

5. The illuminator assembly according to claim 3, wherein the differential-thermal-expansion load applicator is a third compression spring under continuous compression when the illuminator assembly is removably retained in the chamber.

6. The illuminator assembly according to claim 3, wherein the first differential-thermal-expansion compliant conductor is a first compression spring applying a first spring force to the anode pad, the second differential-thermal-expansion compliant conductor is a second compression spring applying a second spring force to the cathode pad, the differential-thermal-expansion load applicator is a third compression spring applying a third spring force to the substrate when the illuminator assembly is removably retained in the chamber and the third spring force is greater than the combination of the first spring force and the second spring force.

7. The illuminator assembly according to claim 3, wherein the emitter is an optical semiconductor device.

8. The illuminator assembly according to claim 3, wherein the emitter is a light emitting diode.

9. The illuminator assembly according to claim 3, wherein the heat sink is thermally coupled to the substrate by an adhesive.

10. The illuminator assembly according to claim 9, wherein the adhesive is in continuous compression.

11. The illuminator assembly according to claim 3, wherein the dental handpiece swivel has an exhaust tube configured to discharge an exhaust fluid into the chamber and the heat sink has an exhaust discharge-tube heat-sink bore through which an exhaust discharge tube extends, the exhaust tube, the exhaust discharge tube and the chamber forming a fluid passageway when the illuminator assembly is removably retained in the chamber.

12. The illuminator assembly according to claim 3, wherein the heat sink has an anode-pin heat-sink bore through which the anode pin extends and a cathode-pin heat-sink bore through which the cathode pin extends

13. The illuminator assembly according to claim 3, wherein the heat sink has an anode-pin heat-sink bore through which the anode pin extends and a cathode-pin heat-sink bore through which the cathode pin extends, a first end of the anode pin is proximal to and spaced from the anode pad, a first anode-pin bore in the first end of the anode pin supports the first differential-thermal-expansion compliant conductor, a first end of the cathode pin is proximal to and spaced from the cathode pad, a first cathode-pin bore in the first end of the cathode pin supports the second differential-thermal-expansion compliant conductor.

14. The illuminator assembly according to claim 13, wherein the anode-pin heat-sink bore has a radially outwardly extending anode-pin heat-sink bore slot and the cathode-pin heat-sink bore has a radially outwardly extending cathode-pin heat-sink bore slot, the first end of the anode pin has a radially outwardly extending anode-pin tab in the anode-pin heat-sink bore slot and the first end of the cathode pin has a radially outwardly extending cathode-pin tab in the cathode-pin heat-sink bore slot.

15. A swivel coupling for coupling a dental handpiece to a dental-handpiece hose assembly comprising:
a housing having a first end configured to removably and swivelably couple to the dental handpiece and a second end configured to removably couple to the dental-handpiece hose assembly;
a chamber in the housing;
an illuminator assembly removably retained in the chamber, the illuminator assembly comprising:
an emitter mounted on a substrate having an anode pad and a cathode pad electrically connected to the emitter;
a first differential-thermal-expansion compliant conductor in continuous electrical contact with the anode pad;
a second differential-thermal-expansion compliant conductor in continuous electrical contact with the cathode pad
a heat sink thermally coupled to the substrate; and
a differential-thermal-expansion load applicator operatively coupled to the substrate and to a surface of the chamber, the differential-thermal-expansion load applicator configured to maintain a thermal interface between the substrate and the heat sink in continuous compression.

16. The swivel coupling of claim 15, wherein the first differential-thermal-expansion compliant conductor has an anode conductor end portion distal to the anode pad, the anode conductor end portion being an anode pin and the second differential-thermal-expansion compliant conductor has a cathode conductor end portion distal to the cathode pad, the cathode conductor end portion being a cathode pin.

17. The swivel coupling of claim 15, further comprising an optical path extending from the chamber.

18. The swivel coupling of claim 17, wherein the optical path comprises a light transmission element.

19. The swivel coupling of claim 18, wherein the light transmission element is a glass rod.
